(19) United States
(12) Patent Application Publication
(10) Pub. No.: US 2017/0333333 A1
(43) Pub. Date: Nov. 23, 2017

(54) Nail Coating Spray

(71) Applicant: LiJuan Zhen, City of Industry, CA (US)

(72) Inventor: LiJuan Zhen, City of Industry, CA (US)

(21) Appl. No.: 15/583,881

(22) Filed: May 1, 2017

Related U.S. Application Data
(60) Provisional application No. 62/330,243, filed on May 1, 2016.

(51) Int. Cl. A61K 8/86; A61K 8/73; A45D 34/00; A61K 8/87; B65D 83/14; A61Q 3/02; A61K 8/19; A61K 8/81

(52) U.S. Cl. CPC A61K 8/86; A61K 8/19; A61K 8/735; A61K 8/8147; A61K 8/87; B65D 83/752; A61Q 3/02; A45D 34/00; A61K 2800/30; A61K 2800/54; A61K 2800/594; A61K 2800/87; A61K 2800/884; A61K 2800/43

(57) ABSTRACT

A peelable nail coating spray comprises at least one film-forming polymer, at least one copolymer, at least one thinner, at least one keratinous nourishing ingredient, and a pressurized container to store the peelable nail coating spray. A method of applying a peelable nail coating spray providing a peelable nail coating spray comprising a at least one film-forming polymer, at least one copolymer, at least one thinner, at least one keratinous nourishing ingredient, and a pressurized container to store the peelable nail coating spray, releasing the peelable nail coating spray from the pressurized container, applying the peelable nail coating spray on a nail surface to form a layer, and peeling off and/or washing off the layer and optionally the second layer from skin or cuticles surrounding the nail surface after the layer is dry.

### CROSS-REFERENCE RELATED TO RELATED APPLICATIONS

This application claims the priority and benefit of U.S. provisional patent application No. U.S. Provisional Application No. 62/330,243, filed May 1, 2016.

### BACKGROUND

Nail polish containing at least one acrylic polymer has been in the market.

Conventionally, the basics of applying nail polish are to apply a first layer of the nail polish directly on nail. The base coat has to be dried to form a base coat. The drying of the base coat can use ultraviolet light source to cure the acrylic polymer, or in some compositions, the first layer of acrylic polymer can be dried in air without cure by ultraviolet light source. The application of the first layer of nail polish on the nail is done by nail brush. The first layer of nail polish is normally transparent or slightly translucent, which cannot be really visualized whether it is applied on nail or sometime over-spilled to cuties.

After the base coat is formed on the nail, a second layer of nail polish can be applied on the base coat. The second layer of nail polish can comprise acrylic polymer or also other copolymer too. The second layer of nail polish can be transparent, translucent, or with color. The application of second layer of nail polish, conventionally, is also by brush. The drying of the second layer can use ultraviolet light source to cure the polymers, or in some compositions, then it can be dried in air without cure by ultraviolet light source.

A third layer of nail polish can be optionally applied on the second layer when the second layer is dried. The third layer can be same nail polish composition as the base coat or the second layer. The third layer is also called top coat, which forms a protective layer on the second layer.

The conventional brush applied nail polish has drawback that the second layer is harder to dry. Also, if the second layer is colored, the strokes of brush often leave marks when the second layer is not evenly applied on the base coat.

U.S. Pat. No. 5,424,121 (“121 Patent”) disclosed a method to apply a color to a nail surface comprising applying a base coat on nail surface, spraying at least one color of acrylic paint on the base coat, and apply a top coat on the second layer. The 121 Patent further disclosed a method to apply a stencil on the base coat and spray a second coat on the stencil. However, the 121 Patent does not teach, suggest, or motivate the details of the compositions of the acrylic paint, solvents, other additives, and how the acrylic paint can become sprayable. The 121 patent also teaches that a base coat shall be applied and be waited for the base coat to dry before applying color acrylic paint. The 121 patent also teaches that a top coat to seal the acrylic paint is required to protect the color. The applications of the base coat to prepare the nail surface and the top coat to seal the color acrylic paint is time consuming and cumbersome.

A U.S. Patent Application Publication No. 2015/0283043 A1 (““043 Application”) disclosed and claimed a sprayable decorative nail polish, comprising a propellant and a decorative liquid polish, which further comprises at least one thinner, in a percentage of 75 to 90%, at least one fixative, in a percentage of 0.5 to 3%, at least one colorant, in a percentage of 5 to 20%, and optionally at least one perfume, in a percentage of 0.1 to 5%, at least one additive, in a percentage of 0.1 to 5%, by weight. Also disclosed by the “043 Application is that the sprayable decorative nail polish is applied on the base coat.

The 043 Application teaches that a base coat applied by brush on nail is required and needs to be utmost care applied to avoid covering the cuticles or the skin with polish base. The “043 Application also teaches that the percentage of the fixative needs to be very small (less than 3%) so the fixative is not sufficient to allow the decorative component to be fixed on parts of the skin and cuticles that are not pretreated with base coat. The “043 application also teaches, as the “121 patent, that the overspray of decorative nail polish on skin or cuticles needs to be washed off. The drawback of the sprayable decorative nail polish disclosed by the “043 Application is that the fixative amount is too low that might not be sufficient to form a durable layer; therefore, a top coat is essential by the method of applying the sprayable decorative nail polish claimed by the “043 Application. The necessary application of the top coat prolongs the application of nail polish and increase the cost. Therefore, it is not a preferable method and composition of nail polish.

Also some traditional nail polishes contain drying oil, such as triglycerides comprising conjugated double bonds have the problem of stability of the nail polish separation of oil and pigment. Also some traditional nail polishes have plasticizer comprising glycol and their derivatives, such as glycol ethyl ether, glycol esters, camphor and/or dibutyl phthalate (DBP) that has been in a controversy linking to cancer in recent years.

Some nail coating product also contain copolymer acrylate for enhancing adhesion. The copolymer acrylate is formed by reaction of acrylate or acrylic monomers, which may cause redness, swelling, and pain in the skin such as cuticle. Therefore, it is desired to solve these issues.

### BRIEF SUMMARY OF THE INVENTION

The above issues have been resolved by the current invention for a composition of peelable nail coating spray. The invention is directed to a peelable nail coating spray comprising at least one film-forming polymer, at least one copolymer, at least one thinner, at least one keratinous nourishing ingredient, wherein the peelable nail coating spray is stored in a pressurized container. The invention is also directed to a method of applying a peelable nail coating spray comprises at least one film-forming polymer, at least one copolymer, at least one thinner, at least one keratinous nourishing ingredient, wherein the peelable nail coating spray is stored in a container, and wherein further comprises the steps of releasing the peelable nail coating spray from the pressurized container, applying the peelable nail coating spray on a nail surface to form a layer, and peeling off and/or washing off the layer and optionally the second layer from skin or cuticles surrounding the nail surface after the layer is dry, but the layer on the cuticles can be washing off too. One can either peel off, wash off, or peel off and wash off the nail coating spray on skin or cuticles. The washing medium is water and optionally with soap.

### DETAILED DESCRIPTION

Before the present invention is described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Other than in the embodiment or example, or where indicated otherwise, all numbers indicating ingredient quantities and/or reaction conditions are to be understood as being modified in every instance by the word “about,” which means the ingredient quantities or reaction conditions are within 10 percent to 15 percent of the indicated value.

Unless defined otherwise, all terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” may also include the plural referents unless the context clearly dictates otherwise.

It is further noted that the claims may be drafted to exclude any element that may be optional. As such, this statement is intended to serve as antecedent basis for use of such exclusive terminology as “solely”, “only” and the like in connection with the recitation of claim elements, or the use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The disclosure is related to a peelable nail coating spray comprising at least one film-forming polymer, at least one copolymer, at least one thinner, at least one keratinous nourishing ingredient, wherein the peelable nail coating spray is stored in a container, and wherein the container can be pressurized. The invention is also directed to a method of applying a peelable nail coating spray comprises at least one film-forming polymer, at least one copolymer, at least one thinner, at least one keratinous nourishing ingredient, wherein the peelable nail coating spray is stored in a container, and wherein further comprises the steps of releasing the peelable nail coating spray from the pressurized container, applying the peelable nail coating spray on a nail surface to form a layer, peeling off and/or washing off the layer and optionally the second layer from skin or cuticles surrounding the nail surface after the layer is dry, but the layer on the cuticles can be washing off too. One can either peel off, wash off, or peel off and wash off the nail coating spray on skin or cuticles. The washing medium is water and optionally with soap.

The peelable is defined as the film or coating formed by polymer on skin or nail after drying is removable without involving the use of polish remover, such as acetone.

In one preferred embodiment of the peelable nail coating spray, wherein the at least one film-forming polymer is selected from the group consisting of acrylic polymer and polyurethanes between about 6% and about 15% by weight relative to the total weight of the peelable nail coating spray. The peelable nail coating spray can be dried between about 20 to about 30 seconds.

In one preferred embodiment of the peelable nail coating spray wherein the at least one copolymer is between about 6% and about 15% by weight relative to the total weight of the peelable nail coating spray. In one preferred embodiment of the copolymer, Bis(Glycidoxyphenyl) propane/Bisaminomethyl norbornane copolymer is used between about 6% and about 15% by weight relative to the total weight of the peelable nail coating spray. The copolymer can provide a smooth surface to reduce bubbles on the nail after spraying the peelable nail coating spray and enhance the forming of the layer.

In one of the embodiments of the peelable nail coating spray, wherein the thinner is a solvent or a monomer between about 50% and about 70% by weight relative to the total weight of the peelable nail coating spray, wherein the solvent is preferably as organic solvent, preferably cosmetic and toxicological suitable for human use, and are either soluble in or miscible with water or insoluble in or immiscible with water. Such solvent includes but not limited to alkyl acetates, alkyl alcohols, alkyl ketones, such as ethyl acetate, isobutyl acetate, butyl acetate, isopropanol, ethanol, methylethy ketone, butyl acetate, ethanol, n-butanol, isopropyl alcohol, isododecane, acetone, acetate, benzyl alcohol, bis-2-methoxyethyl-ether, ethylene carbonate, 1,6-hexanediol, propylene glycol, cyclohexane, n-hexane, amyl acetate, isoamyl acetate, butanol, acetonitrile, and mixture thereof. The monomer is preferably cosmetic and toxicological suitable for human use, and are either hydrophilic, hydrophobic, or amphiphile. Such monomer includes but not limited to acrylic monomers, acrylic containing monomers, vinyl monomers, styrene monomers, and mixture thereof.

In one preferred embodiment of the peelable nail coating spray, wherein the keratinous nourishing ingredient is cosmetically safe, and wherein the keratinous nourishing ingredient is preferable selected but not limited to from the group consisting of vitamin C, vitamin D, d-alpha-tocopherol, magnesium 1-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane -tetracosane, urea perhydrate, bioflavonoids, acetyl tyrosine, lecithin, polymethylsiloxane, glycerol-1,2,3-trihydroxypropane, natural ingredients, extract of fruits, extract of plants, and mixtures thereof, and wherein the natural ingredients are selected from the group consisting of kojic acid, licorice, mulberry, camellia sinensis extract, euterpe oleracea fruit extract, grape seed polyphenols, emollien, oenothera biennis oil, aloe barbadensis juice, hippophae rhamnoides fruit extract, jojoba esters, vitis vinifera grape fruit extract, and punica granatum pomegranate extract, and mixtures thereof. The keratinous nourishing ingredient provide moistures and nutrition to the nail as well as the skin or cuticles. Since peelable nail coating spray contains thinner, which will evaporate in drying of the polish. When the thinner is evaporated, water or moisture on skin, cuticles, or nail will be evaporated too. Therefore, the skin, cuticles, or nails will normally become dry or dehydrated. The current invention is added with keratinous nourishing ingredients, which provides moisture or nutrients to supplement and lock the moisture on the skin, cuticles, or nails. The preferred embodiment of the additive is hyaluronic acid.

In one of the embodiments of the peelable nail coating spray can additionally and optionally further comprising at least one additive, wherein the additive can be pigment for color, pearls, pearlescent, aluminum oxides for sparkling or glittering effect, and white powders for silky effect, and wherein the additive is at least about 0.1° A by weight relative to the total weight of the peelable nail coating spray. The additive is to add color or glittering effects.

In one of the embodiments of the peelable nail coating spray, wherein the pressurized container further comprises pressure source selected from compressed gasses, liquidized gases for pharmaceutical use, and a mixture thereof. The compressed gases, for example, can be nitrogen, nitrous oxide, carbon dioxide, and the mixture thereof. The liquidized gases for pharmaceutical use, for example, can be hydrocarbons. The pressure source can be released from the can and brings out the peelable nail coating spray as aerosols through the nozzle of the pressurized can.

In one of the embodiments of the current invention is also directed to a method of applying the peelable nail coating spray on a nail, comprising at least one film-forming polymer, at least one copolymer, at least one thinner, at least one keratinous nourishing ingredient, and a pressurized container to store the peelable nail coating spray. The method further comprises the steps of releasing the peelable nail coating spray from the pressurized container; applying the peelable nail coating spray on a nail surface to form a layer; optionally applying the peelable nail coating spray on the layer after the layer is dry to form a second layer; and peeling off and/or washing off the layer and optionally the second layer from skin or cuticles surrounding the nail surface after the layer is dry. It is only optional to apply a base coat in the preferred embodiment of applying the peelable nail coating spray on a nail before applying the peelable nail coating spray. In one embodiment of the invention, a base coat is applied to the nail before applying the peelable nail coating spray. It is also only optional to apply a top coat in the preferred embodiment of applying the peelable nail coating spray on a nail after applying the peelable nail coating spray. In one embodiment of the invention, a top coat is applied to the layer formed by the peelable nail coating spray after the peelable nail coating spray is dried.

According to the present invention, it is not necessary to apply a base coat before apply the peelable nail coating spray on nail. Also, it does not need to apply a top coat to seal the peelable nail coating spray on nail. A second layer of peelable nail coating spray can be applied on the first layer of peelable nail coating spray on nail. Therefore, the current invention has resolved the need to have an utmost care to apply a base coat on nail. Also, it shorten the time to apply nail coating spray by eliminating the time spent on applying and waiting of base coat.

The present invention is also against the teaching of others that the overspray on skin or cuticles needs to be removed only by washing the skin or cuticles. In the preferred embodiment of the invention, the overspray of the present invention on skin is removed from the skin or cuticles by peeling off, washing off, or by peeling off and washing off the film formed by the peelable nail coating spray after the layer is dried. In one of the embodiments of the method applying the peelable nail coating spray, the peelable nail coating spray on skin or cuticles is washed and/or peeled off from the skin or cuticles. The washing medium is water and optionally with soap.

Having described the subject matter of the present disclosure detailed description of embodiments and examples for purposes of clarity of understanding to a person with ordinary skill in the art that the same can be performed by modifying or changing the subject matter within various conditions, by various formulations and by other parameters without affecting its scope or any specific embodiment thereof, and that such modifications or changes are intended to be encompassed within the scope of the claims.

What is claimed:

1. A peelable nail coating spray comprising
at least one film-forming polymer;
at least one copolymer;
at least one thinner;
at least one keratinous nourishing ingredient; and
a pressurized container to store the peelable nail coating spray.

2. The peelable nail coating spray of claim 1, wherein the at least one film-forming polymer is selected from the group consisting of acrylic polymer and polyurethanes between about 6% and about 15% by weight relative to the total weight of the peelable nail coating spray.

3. The peelable nail coating spray of claim 1, wherein the at least one copolymer is between about 6% and about 15% by weight relative to the total weight of the peelable nail coating spray.

4. The peelable nail coating spray of claim 1,
wherein the at least one copolymer comprises Bis(Glycidoxyphenyl) propane/Bisaminomethyl norbornane copolymer.

5. The peelable nail coating spray of claim 1, wherein the at least one thinner is between about 50% and about 70% by weight relative to the total weight of the peelable nail coating spray, wherein the thinner is selected from the group consisting of solvent, monomer, and mixture thereof.

6. The peelable nail coating spray of claim 1, wherein the keratinous nourishing ingredient is cosmetically safe, and wherein the keratinous nourishing ingredient is selected from the group consisting of hyaluronic acid, amino acids, aminoacetic acid, vitamins, d-alpha-tocopherol, magnesium l-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane tetracosane, urea perhydrate, bioflavonoids, acetyl tyrosine, lecithin, polymethylsiloxane, glycerol 1,2,3-trihydroxypropane, natural ingredients, extract of fruits, extract of plants, and mixtures thereof, and wherein the natural ingredients are selected from the group consisting of kojic acid, licorice, mulberry, camellia sinensis extract, euterpe oleracea fruit extract, grape seed polyphenols, emollien, oenothera biennis oil, aloe barbadensis juice, hippophae rhamnoides fruit extract, jojoba esters, vitis vinifera grape fruit extract, and punica granatum pomegranate extract, and mixtures thereof.

7. The peelable nail coating spray of claim 1, wherein the keratinous nourishing ingredient is cosmetically safe, and wherein the keratinous nourishing ingredient is hyaluronic acid.

8. The peelable nail coating spray of claim 1, wherein the pressurized container further comprises a pressure source in the pressurized container selected from the group consisting of a compressed gas, liquidized gas for pharmaceutical use, and a mixture thereof.

9. The peelable nail coating spray of claim 1, wherein the compressed gas comprises a compressed nitrogen gas.

10. The peelable nail coating spray of claim 1, wherein the peelable nail coating spray is devoid of drying oil, and wherein the peelable nail coating spray is devoid of plasticizer agent.

11. The peelable nail coating spray of claim 1, further comprising at least one additive.

12. The peelable nail coating spray of claim 11, wherein the additive can be pigment for color, pearls, pearlescent, aluminum oxides for sparkling or glittering effect, and white powders for silky effect, and wherein the additive is at least about 0.1% by weight relative to the total weight of the peelable nail coating spray.

13. A peelable nail coating spray comprising
at least one film-forming polymer;
at least one copolymer;
at least one thinner;
at least one keratinous nourishing ingredient;
a pressurized container to store the peelable nail coating spray,
wherein the at least one film-forming polymer is selected from the group consisting of acrylic polymer and polyurethanes between about 6% and about 15% by weight relative to the total weight of the peelable nail coating spray,
wherein the at least one copolymer is between about 6% and about 15% by weight relative to the total weight of the peelable nail coating spray,
wherein the at least one thinner is between about 50% and about 70% by weight relative to the total weight of the peelable nail coating spray, wherein the thinner is selected from the group consisting of solvent and monomer, and mixture thereof,
wherein the keratinous nourishing ingredient is cosmetically safe, and wherein the keratinous nourishing ingredient is hyaluronic acid, and
wherein the pressurized container further comprises a pressure source a pressure source of the pressurized container selected from the group consisting of a compressed gas, liquidized gas for pharmaceutical use, and a mixture thereof.

14. The peelable nail coating spray of claim 13, further comprising at least one additive,
wherein the at least one copolymer comprises Bis(Glycidoxyphenyl) propane/Bisaminomethyl norbornane copolymer,
wherein the compressed gas comprises a compressed nitrogen gas, and
wherein the additive can be pigment for color, pearls, pearlescent, aluminum oxides for sparkling or glittering effect, and white powders for silky effect, and wherein the additive is at least about 0.1% by weight relative to the total weight of the peelable nail coating spray.

15. A method of applying a peelable nail coating spray comprising:
providing a peelable nail coating spray, wherein the peelable nail coating spray comprises at least one film-forming polymer, at least one copolymer, at least one thinner, at least one keratinous nourishing ingredient; and a pressurized container to store the peelable nail coating spray;
releasing the peelable nail coating spray from the pressurized container toward at least one nail surface;
applying the peelable nail coating spray on the at least one nail surface to form a layer;
optionally applying the peelable nail coating spray on the layer after the layer is dry to form a second layer; and
peeling off and/or washing off the layer and optionally the second layer from skin or cuticles surrounding the nail surface after the layer is dry.

16. The method of claim 15,
wherein the peelable nail coating spray further comprises at least one additive,
wherein the at least one film-forming polymer is selected from the group consisting of acrylic polymer and polyurethanes between about 6% and about 15% by weight relative to the total weight of the peelable nail coating spray,
wherein the at least one copolymer is between about 6% and about 15% by weight relative to the total weight of the peelable nail coating spray,
wherein the at least one thinner is between about 50% and about 70% by weight relative to the total weight of the peelable nail coating spray,
wherein the thinner is selected from the group consisting of solvent and monomer, and mixture thereof,
wherein the keratinous nourishing ingredient is cosmetically safe,
wherein the keratinous nourishing ingredient is hyaluronic acid,
wherein the additive can be pigment for color, pearls, pearlescent, aluminum oxides for sparkling or glittering effect, and white powders for silky effect, and wherein the additive is at least about 0.1% by weight relative to the total weight of the peelable nail coating spray, and
wherein the pressurized container further comprises a pressure source a pressure source of the pressurized container selected from the group consisting of a compressed gas, liquidized gas for pharmaceutical use, and a mixture thereof.

17. The method of claim 15,
wherein the peelable nail coating spray further comprises at least one additive,
wherein the at least one film-forming polymer is selected from the group consisting of acrylic polymer and polyurethanes between about 6% and about 15% by weight relative to the total weight of the peelable nail coating spray,
wherein the at least one copolymer is between about 6% and about 15% by weight relative to the total weight of the peelable nail coating spray,
wherein the at least one thinner is between about 50% and about 70% by weight relative to the total weight of the peelable nail coating spray,
wherein the thinner is selected from the group consisting of solvent and monomer, and mixture thereof,
wherein the keratinous nourishing ingredient is cosmetically safe,
wherein the keratinous nourishing ingredient is selected from the group consisting of hyaluronic acid, amino acids, aminoacetic acid, vitamin, d-alpha-tocopherol, magnesium 1-ascorbyl-2-phosphate, ferulic acid, ascorbic acid, ubiquinone, allantoin, 2,5-dioxo-4-imidazolidinyl, 2,3-dihydroxypropyl ester octadecanoic acid, superoxide dismutase, tocopheryl acetate, squalane tetracosane, urea perhydrate, bioflavonoids, acetyl tyrosine, lecithin, polymethylsiloxane, glycerol 1,2,3-trihydroxypropane, natural ingredients, extract of fruits, extract of plants, and mixtures thereof, and wherein the natural ingredients are selected from the group consisting of kojic acid, licorice, mulberry, camellia sinensis extract, euterpe oleracea fruit extract, grape seed polyphenols, emollien, oenothera biennis oil, aloe barbadensis juice, hippophae rhamnoides fruit extract, jojoba esters, vitis vinifera grape fruit extract, and punica granatum pomegranate extract, and mixtures thereof,
wherein the additive can be pigment for color, pearls, pearlescent, aluminum oxides for sparkling or glittering effect, and white powders for silky effect, and wherein the additive is at least about 0.1% by weight relative to the total weight of the peelable nail coating spray, and
wherein the pressurized container further comprises a pressure source a pressure source of the pressurized container selected from the group consisting of compressed gasses, liquidized gases for pharmaceutical use, and a mixture thereof.

18. The method of claim 15,
wherein the at least one copolymer comprises Bis(Glycidoxyphenyl) propane/Bisaminomethyl norbornane copolymer and
wherein the compressed gas comprises a compressed nitrogen gas.
